(19) United States
(12) Patent Application Publication
(10) Pub. No.: US 2026/0083456 A1
(43) Pub. Date: Mar. 26, 2026

(54) AORTIC DISSECTION AND AORTIC FALSE LUMEN EMBOLIZATION DEVICE

(71) Applicant: SHAPE MEMORY MEDICAL, INC., San Jose, CA (US)

(72) Inventors: Le LE, San Jose, CA (US); Chung YEH, San Mateo, CA (US); Landon D. NASH, Park City, UT (US); Todd L. LANDSMAN, Ojai, CA (US); Sayyeda Marziya HASAN, Los Gatos, CA (US); Kasey KWONG, Portland, OR (US); Stewart KUME, Emerald Hills, CA (US); Rochelle MARLANGAUE, Fremont, CA (US); Thai NGUYEN, Santa Clara, CA (US)

(21) Appl. No.: 18/873,381

(22) PCT Filed: Jun. 29, 2023
(86) PCT No.: PCT/US2023/026547

§ 371 Date: Dec. 10, 2024

Related U.S. Application Data
(60) Provisional application No. 63/356,598, filed on Jun. 29, 2022.
(60) Provisional application No. 63/383,665, filed on Nov. 14, 2022.
(60) Provisional application No. 63/510,662, filed on Jun. 28, 2023.

(51) Int. Cl. A61B 17/12; A61B 17/00; A61B 90/00; A61L 31/06; A61L 31/14

(52) U.S. Cl. CPC A61B 17/12118; A61B 17/12163; A61B 17/12181; A61B 90/03; A61B 90/39; A61L 31/06; A61L 31/146; A61B 2017/00871; A61B 2017/12054; A61B 2090/031; A61B 2090/3966

(57) ABSTRACT

An embodiment includes a system comprising: a backbone; a first polyurethane shape memory polymer (SMP) foam on the backbone; a second polyurethane SMP foam on the backbone; a third polyurethane SMP foam on the backbone and between the first and second SMP foams. Other embodiments are described herein.

### CROSS-REFERENCE TO RELATED APPLICATIONS

This application claims priority to U.S. Provisional Patent Application No. 63/383,665 filed on Nov. 14, 2022 and entitled “Aortic Dissection and Aortic False Lumen Embolization Device”, the content of which is hereby incorporated by reference. This application further claims priority to U.S. Provisional Patent Application No. 63/356,598 filed on Jun. 29, 2022 and entitled “False Lumen/Aortic Dissection Therapeutic Device”, the content of which is hereby incorporated by reference. This application further claims priority to U.S. Provisional Patent Application No. 63/510,662 filed on Jun. 28, 2023 and entitled “False Lumen/Aortic Dissection Therapeutic Device”, the content of which is hereby incorporated by reference.

### BACKGROUND

Aortic dissection (AD) occurs when an injury to the innermost layer of the aorta allows blood to flow between the layers of the aortic wall, forcing the layers apart. In most cases, this is associated with a sudden onset of severe chest or back pain, often described as “tearing” in character. Also, vomiting, sweating, and lightheadedness may occur. Other symptoms may result from decreased blood supply to other organs, such as stroke, lower extremity ischemia, or mesenteric ischemia. AD can quickly lead to death from insufficient blood flow to the heart or complete rupture of the aorta.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIGS. 1A-1F illustrate an embodiment of a method of treating AD with an embodiment of an AD system.

FIGS. 2A-2C illustrate “pearls on a string” foams on a backbone in an embodiment.

FIGS. 3A-3D illustrate various implant foam cross-sections for embodiments.

FIGS. 4A-4C depict various delivery configurations of embodiments.

FIGS. 5A-5D depict an embodiment of a method for applying torque to an embodiment of an AD system.

FIGS. 6A-6E illustrate various embodiments of multi-foam implants.

FIGS. 7A-7D illustrate various foam implant nesting embodiments (e.g., counter bore). FIGS. 8A-8B show the embodiments in greater detail.

FIGS. 9A-9B illustrate various foam implant embodiments wherein the foams have varying expanded foam diameters. FIG. 9C depicts spacing between various foams but a coupling between an end cap foam and an adjacent foam.

FIG. 9D depicts an arrangement that, in at least some instances, is undesirable and prevented by the embodiment of FIG. 9C.

FIGS. 10A-10B illustrate foam embodiments that may include one or more slidable foams or foam portions.

FIGS. 11A-11B illustrate foam embodiments that may include one or more slidable foams or foam portions along with UV adhesive and/or radiopaque marker bands.

FIGS. 12A-12B and 13A-13B include various embodiments of foams with tapered ends due to marker bands compressing portions of the foams.

FIGS. 14A-14B depict an embodiment of a method utilizing axial shortening/clinching.

FIGS. 15A-15D depict an embodiment of a method utilizing axial shortening/clinching.

FIGS. 16A-16B depict an embodiment that utilizes entanglement to occlude a void such as the false lumen of an AD.

FIGS. 17A-17D depict various embodiments of systems utilizing tear-away sleeves that enclose foams.

FIGS. 18A-18B depict various embodiments utilizing removable delivery wires to promote imaging during system delivery.

FIGS. 19A-19D depict various embodiments using delivery catheters with flexible tubes for contrast injection.

FIGS. 20A-20D depict various attachment/detachment embodiments for AD systems.

FIGS. 21A-21F depict various embodiments that promote smaller crimp diameter AD systems.

FIGS. 22A-22J depict various anchoring embodiments for AD systems.

FIGS. 23A-23E depict various anchoring embodiments for AD systems.

FIG. 24 depicts an embodiment of a delivery device for AD systems.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

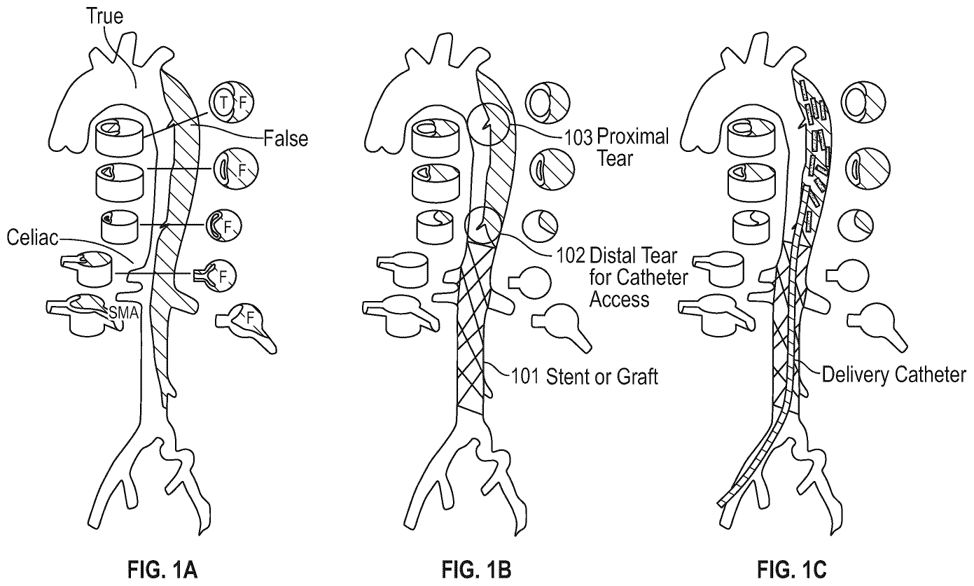

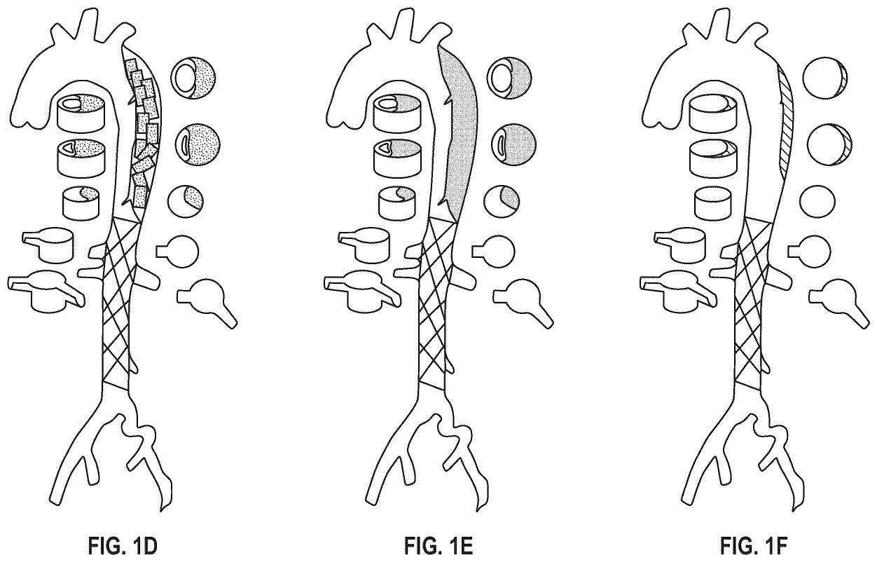

An embodiment is a shape memory polymer foam embolization implant to treat AD and false lumens in the aorta. ADs present very complex, patient-specific anatomies that are difficult to treat. There are typically multiple fenestrations/tears along the length of the AD that interact with other vessels branching from the aorta (e.g., renal arteries, SMA, Celiac artery, etc.). An embodiment may be delivered through tears or fenestration(s) of the false lumen wall (e.g., proximal tear 103 or distal tear 102), and delivered into the false lumen space in a compressed, minimally invasive form factor. To achieve access to the false lumen space, a catheter 104 may be introduced to the femoral artery, and guided through a distal exit tear in the descending aorta section of the dissection (FIG. 1C). Once delivered to the false lumen anatomy, the device expands to volumetrically fill the false lumen space and depressurize the false lumen (FIG. 1D). This depressurization (through thrombus formation) stabilizes the inflow/outflow of the false lumen (FIG. 1E). The porous geometry of the implanted foam device effectively provides volumetric thrombosis within the false lumen and restricts flow in complicated fenestrated anatomies. The expanded material is highly compliant and reduces the risk of further dissection or rupturing of the vessel anatomy. The embolization device can be used in conjunction with other endovascular techniques (e.g., stent 101, graft, etc.) to prepare the AD anatomy for optimal embolization implant delivery.

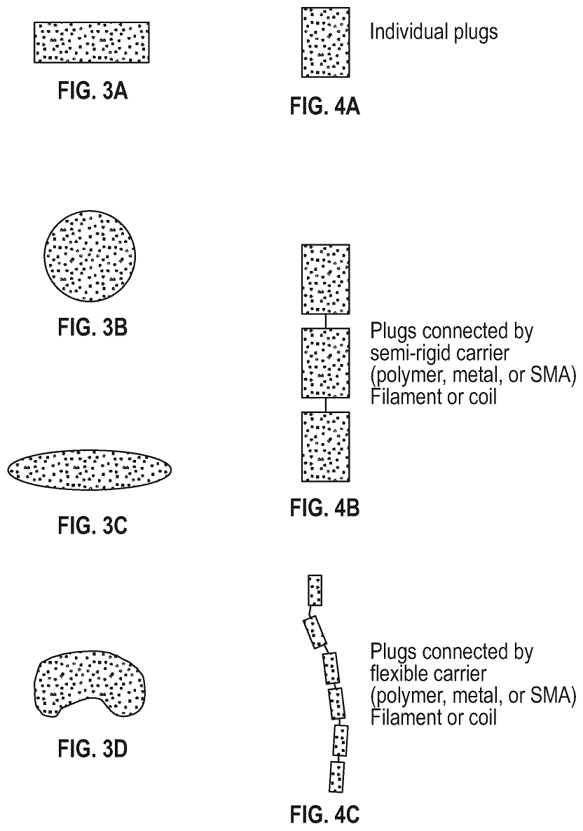

The implant cross section can be, but is not limited to, circular, rectangular, or kidney shapes, as seen in FIGS. 3A-3D. The implant can be comprised of multiple individual plugs, such as the plug of FIG. 4A. In an embodiment 3, 4, 5 or more of the plug of FIG. 4A may be deployed in a false lumen. Further, multiple plugs may be attached along a carrier member (semi-rigid or flexible). In FIG. 4B, plugs are connected by a semi-rigid carrier (e.g., polymer, metal, or SMA), filament, coil, and the like. In FIG. 4C, plugs are connected by flexible carrier (e.g., polymer, metal, or SMA), filament, coil, and the like. The carrier member can be composed of any combination of platinum/tungsten/iridium or stainless-steel wires, marker bands, coils, or beads. Carrier members can also be shape memory alloys or shape memory polymers including nitinol or polyurethane. Other flexible carrier member options include, but are not limited to, polypropylene, polyethylene, polysulfone, polyurethane, PEEK, polyester (PLLA/PGA), polyanhydride, PDO, PCL, liquid crystal polymers, or any combination thereof.

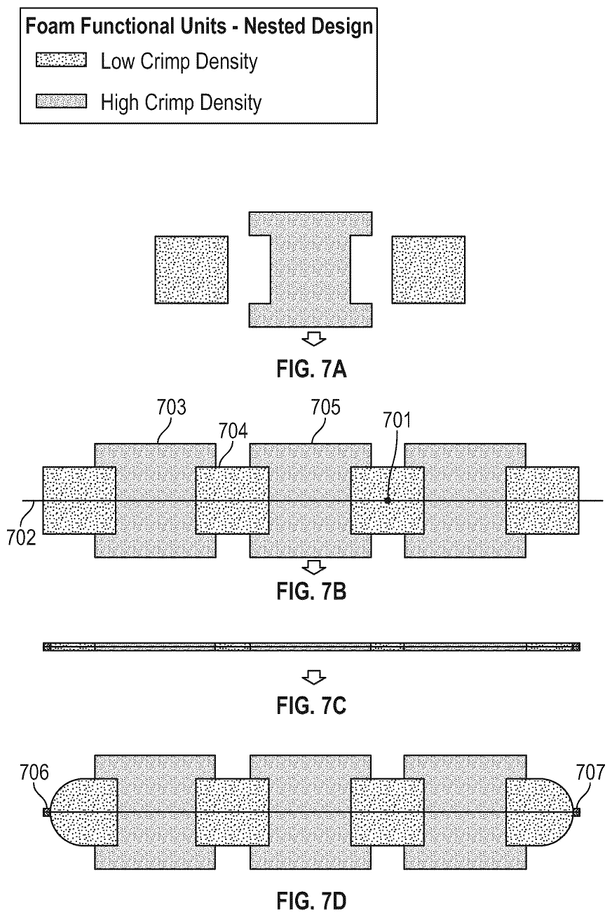

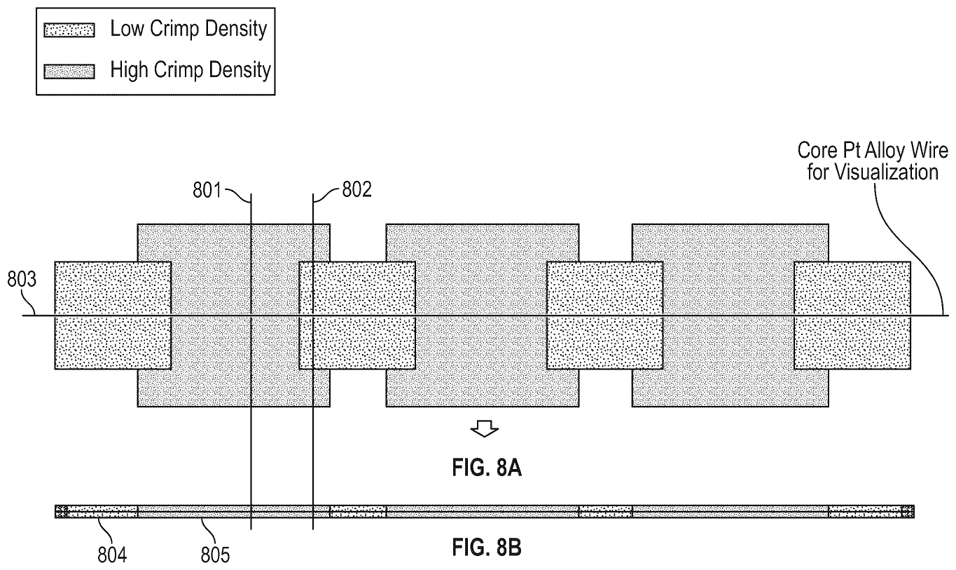

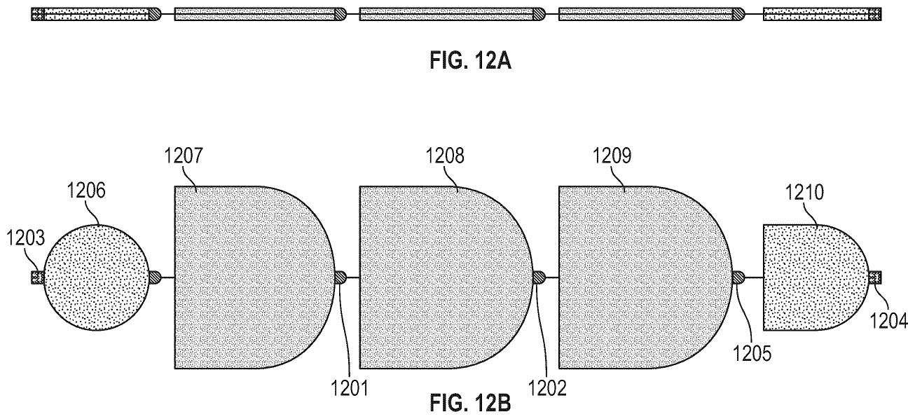

The crimped device profile can be proximally terminated with a dome, fillet, or chamfered geometry to promote axial device alignment when pulling the device proximally back into the delivery catheter during device retrieval. See, for example, FIG. 7D or 12B.

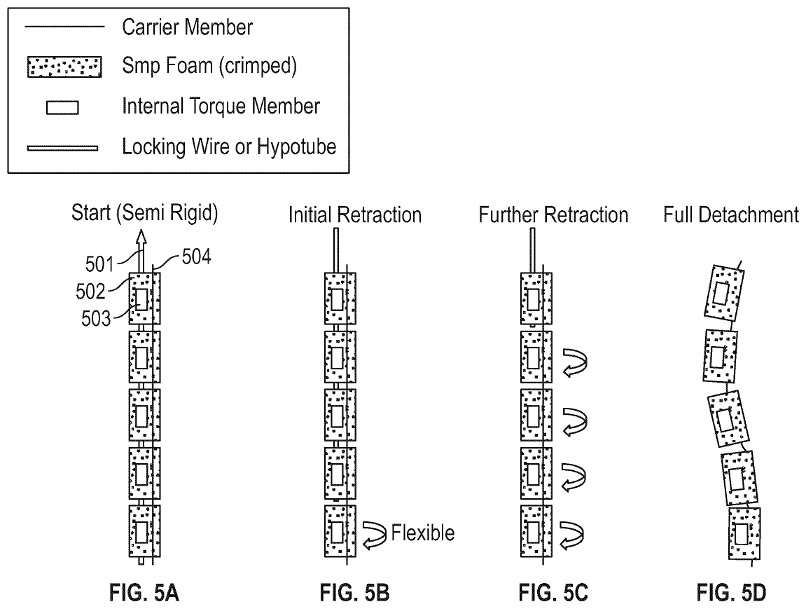

Delivery of flexible carrier member implants, or individual implants, can occur in a random configuration. Alternatively, semi-rigid carrier member implants can be delivered with radial torque control relative to the aorta axis (see, for example, FIGS. 5A-5D). This allows control of non-circular device cross-sections (see, for example, FIGS. 3A-3D) relative to the false lumen cross section. A non-circular device cross-section can help reduce the overall false lumen volume after device implantation and allow for true lumen expansion once the false lumen is depressurized.

In an embodiment, the radial torque control of implant sections can be selectively deactivated by retracting a locking core wire 501 or hypotube (FIGS. 5A-5D). The implant sections contain internal locking torque members 503 that engage with the locking core wire to enable torsion through mechanical interactions (such as lock and key). In this design embodiment, the full length of the device can be axially torqued until the locking core wire is retracted. As the core wire is retracted, the most distal implant section becomes radially flexible and conforms to the anatomy it was delivered in, but proximal implant sections still engaged with the locking core wire can still be torqued (FIG. 5B). Implant sections are sequentially dis-engaged from the locking core wire as it is retracted until the full length of all implant sections are flexible (FIG. 5C). The locking core wire can be independent of the carrier member 504 that connects the implant sections post-implantation. The locking core wire can be radiopaque to identify the axial location of plugs during implant delivery and prior to radiopaque locking core wire retraction. Upon radiopaque locking core wire retraction, the implant could be completely radiolucent to minimize subsequent imaging artifacts, or include radiopaque features (such as marker bands) for device identification via imaging. Marker bands may include, for example, a radiopaque metal band that encircles a foam, wire, and the like.

In an embodiment, the locking core wire 501 can be a semi-rigid elastic or superelastic material such as stainless steel or nitinol, or a combination thereof. The locking core wire can be a wire, tube, coil, ground profile wire, or any combination thereof. The internal locking or torque member 503 may be a metal, polymer, or the like. For example, the internal locking member may be SMP foam. The internal locking member SMP foam may have a density unequal (e.g., greater) than that of the general embolic SMP foam 502 that surrounds the internal locking member. The locking member (regardless of its material composition) may have a square cross-section channel that receives the locking core wire having a similar square cross-section. Rotation of the locking core wire about its long axis would also rotate internal locking member and its embolic SMP foam (which may substantially surround the internal locking member in a plane orthogonal to the long axis of the locking core wire). In other embodiments, the locking core wire may be planar to some extent. For example, similar to a “dipstick” used to check oil in an engine. Such a locking core wire may be disposed into a channel of the internal locking member that has a similar profile (and therefore rotation of the locking core wire will necessarily rotate the internal locking member). In other embodiments, the internal locking member may be omitted and the embolic foam(s) itself may include a slot to receive the flattened locking wire. Due to the strength of the embolic foam, twisting the locking core wire will cause the embolic foam to rotate without damaging the embolic foam.

Once delivered to the false lumen anatomy, the device provides acute hemostasis. Chronically, the implanted shape memory polymer foam serves as a tissue scaffold to promote healing and collagenous scar formation. This contractile collagenous scar tissue promotes lesion shrinkage to further reduce the false lumen volume and promote healthy, true lumen aortic flow volume (FIG. 1F). Over time, the implanted shape memory polymer material biodegrades to leave more full-volume native tissue in the original false lumen volume.

An embodiment of any of the foams described herein include a shape memory polymer foam manufactured from a combination of some or all of N,N,N′,N′-Tetrakis(2-hydroxypropyl)ethylenediamine (HPED), triethanolamine (TEA), hexamethylene diisocyanate (HDI), trimethyl hexamethylene diisocyanate (HDI), 1,2,6-hexanetriol (HT), 3-methyl-1,5-pentanediol (MPD), 2-butyl-2-ethyl propanediol (BEP), 2-Methyl-2,4-pentanediol, or other aliphatic diisocyanates and aliphatic diols or polyols. Other synthetic monomers include ethylene glycol diols and triiodo benzene containing diols or alcohols.

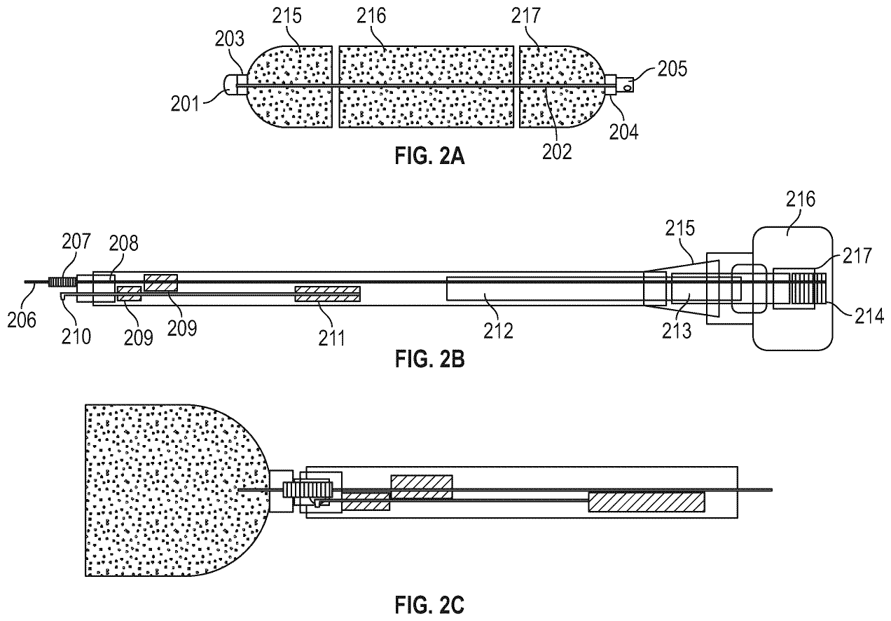

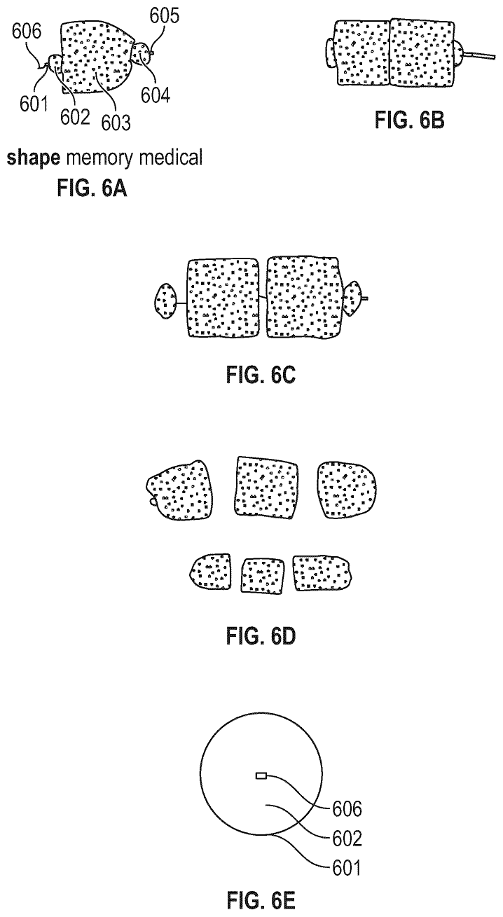

Embodiments may include one or more implant plugs threaded onto a small gauge backbone (e.g., a wire or coil backbone including platinum, iridium, or combinations thereof or a polymer backbone including polyurethane, etc.). FIG. 6A includes an embodiment with three foams 602, 603, 604 along wire 606. Proximal and distal “end caps” 601, 605 are located on either end of the foams. Foams 602, 604 are smaller diameter foam plugs in comparison to foam 603. However, in other embodiments foams 602, 604 could be equivalent in diameter to 603. Elements 601, 605 may be a band (an open cylinder) or a cap (a cylinder with a mostly closed end but for a void to receive a backbone, similar to a bucket with a hole in the bottom of the bucket through which the backbone passes). Such an element may be radiopaque to help a clinician mark proximal and distal bound of the implant. A small backbone may extend through each of the end caps as well as all the foams located between the end caps. The wire may form an axis. As shown in FIG. 6E, a plane orthogonal to that axis may intersect an endcap resulting in a cross-section with cap 601 surrounding a portion of foam 602 and that foam portion surrounding wire 606. The end cap may secure any foam plug or plugs that are between two end caps. Such foam(s) may be fixedly coupled to the backbone (which may include a metal or polymer strand) via an adhesive. However, in other embodiments one or more foams between two end caps may be slidingly coupled to the backbone. For example, the term “pearls on a string” depicted in FIGS. 2A-2C and 6A-6E address embodiments where one or more “pearls” (i.e., foams) slide along a backbone. Embodiments of FIGS. 6A-6E include foams that collectively extend, 20, 30, 40, 50, 60, 70, 80, 90, 100 mm or more along a backbone. Such embodiments include foams with expanded outer diameters of 8, 12, 16, 20, 24, 28, 32, 36, 40, 60 mm or more. FIG. 6C shows four foams on a backbone but FIG. 6D shows two sets of three foams that may be located on a backbone.

Regarding FIGS. 2A-2C, the embodiment of these figures is similar to the embodiments of FIGS. 6A-6E. An embodiment includes SMP foams 215, 216, 217 between platinum/iridium alloy marker bands 203, 204 and adhesive 201 (used to round off distal end of the implantable device). Backbone 202 may include platinum/iridium alloy coil which may be resistant to stretching in some embodiments but which may have the ability to contract under stimulus in other embodiments. The implantable may detach at collar 205 from a push rod, guide wire, wire 210, and the like. Wire 210 may have a projection to fit within an aperture of collar 205. Hypotubes 209 may be used to control the path of wire 210. A series of hypotubes 211, 212, 213 may couple to luer 215, Touhy Borst adaptor with silicon seal 216, stopper slug hypotube 217, termination locking coil 214 to help deliver the SMP foams to the target implantation location. Other delivery systems are suitable in various embodiments, such as the systems described in PCT Patent Publication WO2022040490.

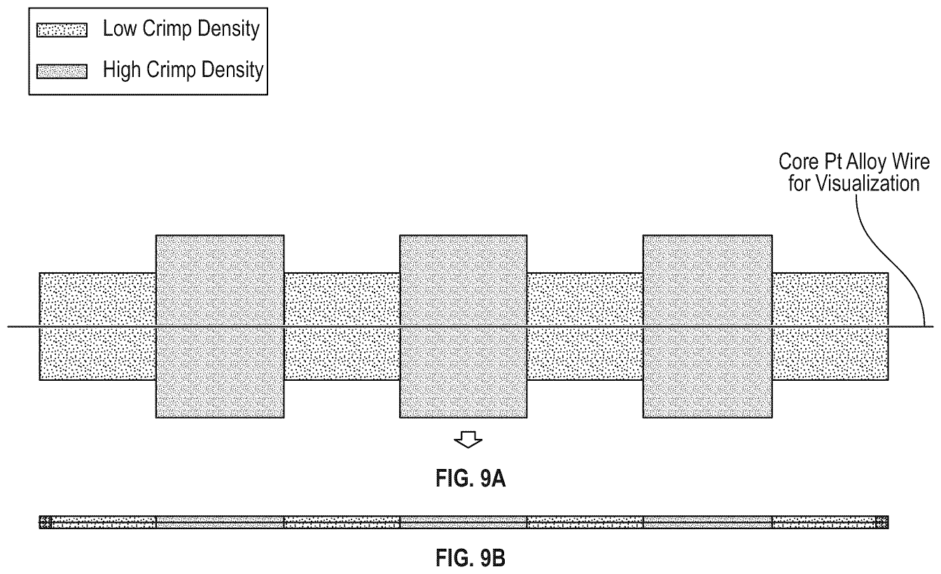

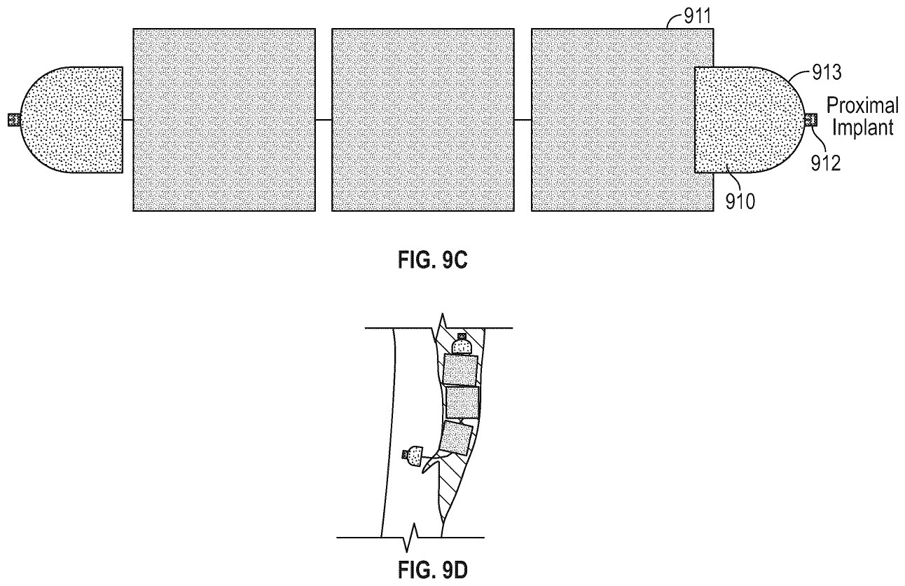

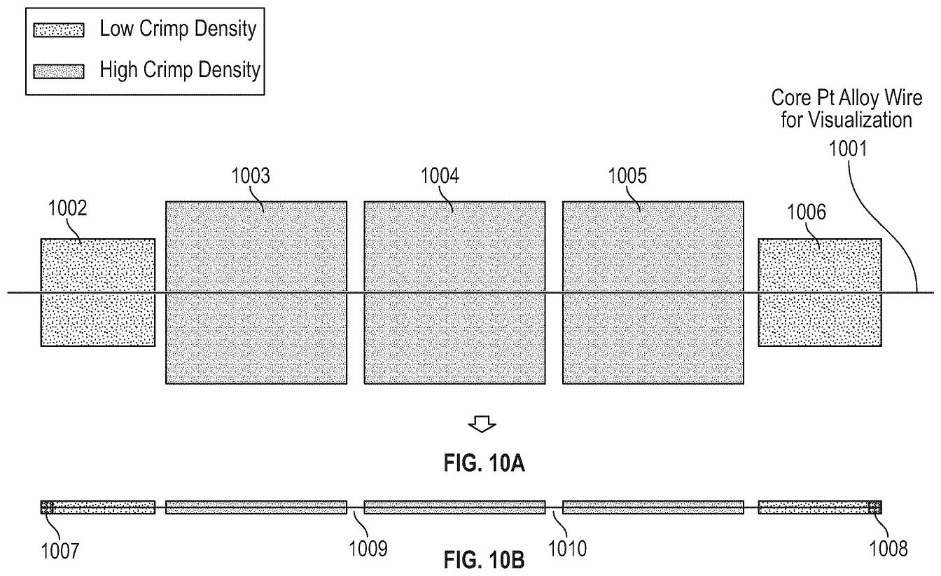

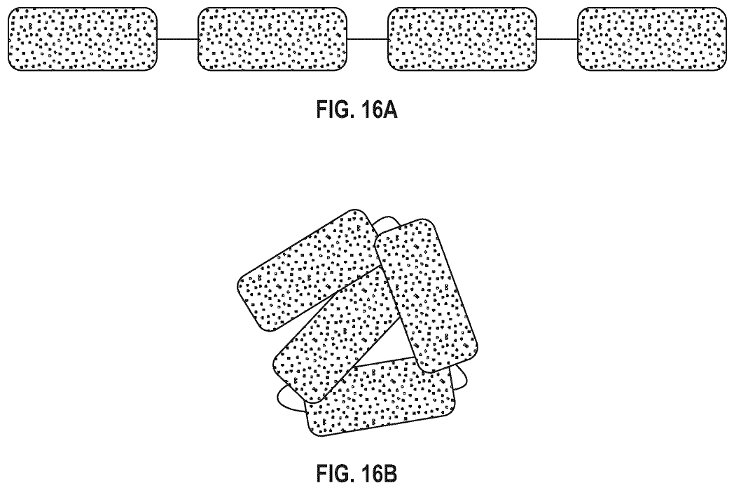

In an embodiment with two or more foams on the backbone, a space or gap between two adjacent foams provides flexibility of the implant and/or axial compliance of the implant structure following deployment. For example, see FIGS. 9C, 10A, 11A, 12B, 13B, 16A. The foams may be delivered in a compressed state (e.g., FIGS. 10B, 11B, 12A, 13A) wherein they have limited flexibility (as opposed to relatively increased flexibility when the foams expand). In their less flexible, compressed state a gap between foams promotes flexibility, which may promote ease of implantation as the device traverse a patient's vasculature. Further, the gaps may facilitate “entanglement” of implant segments (e.g., FIG. 16B) for stability upon final deployment within a patient. However, in other embodiments spaces between foams are not present, thereby promoting the ability to withdraw foams into a deliver conduit without snagging of foams on the conduit entrance/exit orifice. See, for example, FIGS. 7B, 7D, 8A, 9A.

The use of variably sized foams has advantages. For example, FIGS. 6B and 6C show smaller foams proximal and distal to larger foams. A smaller foam may provide more space for endcaps to be affixed to the backbone. An endcap may include a radiopaque collar or bead. The collar or cap may fit over a portion of the smaller foam. A smaller foam may more easily crimp within the endcap. Further, a smaller foam (as opposed to a larger foam) may eventually expand and be less likely (as compared to larger foams) to expand back over the endcap, thereby obscuring the radiopaque character of the end cap.

FIGS. 7A-7D address a “nested” embodiment whereby a portion of each end foam is located within the adjacent larger plug. FIG. 7A shows two smaller foams that are configured to fit into counterbores or voids of the larger foam. The foams 703, 704, 705 are threaded onto core wire 702. FIG. 7B shows how a series of nested foams may be formed on a backbone while using counterbores. FIG. 7C shows the implant with compressed foams and FIG. 7D shows radiopaque marker bands 706. The use of low crimp density foams 704 between high crimp density foams 703, 705 promotes axial and radial flexibility when the implant is being deployed. As used herein, the added radial flexibility provides an increased ability to rotate about, for example, axis 701 (shown “coming out” of the page). Further, despite the varying crimp densities the small and large foams collectively provide an even, consistent outer diameter to help promote possibly withdrawing the implant into a delivery conduit (i.e., avoid a larger diameter portion of the foam failing to easily be withdrawn into a delivery conduit in the event of an initially misplaced implant). FIG. 7D shows the implant after expansion. Note how the marker bands 706 have compressed a portion of the small foams so as those small foams expand, they form edges that taper towards the end cap (e.g., marker band). This rounding of the proximal and distal edges may promote, for example, ease of deployment or recapture of foams into a deliver conduit (e.g., catheter, hypotube, etc.). While in this embodiment the areas of low crimp density foam are associated with smaller foams and high crimp density are associated with larger foams, in other embodiments the foams may have the same outer diameter when expanded yet nevertheless have varying crimp densities.

Regarding figures such as FIGS. 7A-7C, a legend is included that provides hash patterns for low and high crimp density foams. For example, FIG. 7C shows crimped foams. Those hash patterns may also be seen in the expanded foams, such as those in FIG. 7B. Regardless, the legend is applicable to the crimped foams, not the expanded foams. For example, in FIG. 7B the expanded foams may all have the same density, regardless of part diameter and regardless of any differing hash pattern added by a draftsperson. The crimp density (FIG. 7C) is different when you compress those variable diameters to a smaller dimension. So, expanded foams have the lowest density, crimps of smaller diameter plugs (e.g., foam 704) may have medium density, and crimps of large diameter plugs (e.g., foam 705) may have high density. This comment on the figures also applies to the legends for FIGS. 8A-11B.

FIGS. 8A-8B show the embodiment of FIGS. 7A-7C but in greater detail. Note how in FIG. 8B the areas of high crimp density 804 (adjacent low-density area 805) may have densities based on only the large foam (see plane 801) or both the large foam and the small foam (see plane 802). Further, FIG. 8B illustrates the uniform outer diameters of the foams which may facilitate exit out of the delivery conduit or withdrawal back into the delivery conduit. In an embodiment, the entire backbone 803 is radiopaque due its material composition (e.g., platinum alloy).

Further regarding FIG. 8A, the “nested” section of reduced diameter foam may serve as a bridge between central larger diameter plugs with the purpose or eliminating/reducing gaps and providing a contiguous foam segment to facilitate implant retraction. Thus, the embodiment of FIGS. 8A-8B provide: (1) improved axial flexibility due to portion 805, (2) uniform implanted diameter as shown in FIG. 8B, and (3) improved retraction performance (minimized foam shearing) due to uniform implanted diameter.

FIGS. 9A-9B illustrate alternating large and small foams, albeit without counterbores (compared with counterbores in FIG. 8A) in the large foams. Due to the presence of the small foams, despite the lack of counterbores, the implant still has improved axial and radial flexibility and retains the uniform implanted diameter (i.e., before expansion) addressed in FIGS. 7A-7D. However, this embodiment may provide ease of manufacturing as compared to embodiments with counterbores.

The embodiment of FIGS. 9A-9B provide: (1) improved axial flexibility due to portion 905, (2) uniform implanted diameter as shown in FIG. 9B, and (3) improved retraction performance (minimized foam shearing) due to uniform implanted diameter.

An embodiment may include both nested adjacent foams and non-nested adjacent foams. The ability to use either technique provides an ability to balance needs for flexibility versus more controlled axial positioning of the plugs. For example, a distal region of the implant may benefit from fixed foams that are not nested with each other while a proximal region of the implant (configured for implantation near an entry to a void, such as an aortic aneurysm or AD) may have foams that slide and may bunch against one another to better seal the void entrance. The bunching and sealing may be promoted by the use of nested foams. Even if the non-nested foams do not slide, the lack of nesting may promote flexibility.

In FIG. 9C the proximal end of the implant is constructed with the end-cap foam 910 (most proximal foam) nested within the first large diameter plug 911. This facilitates stability and tissue apposition of the proximal portion of the implant after foam expansion. Radiopaque band 912 may fit around a portion of foam 910 to cause the curving of surface 913. This may also mitigate against the large proximal foam plug expanding within the tissue space, but leaving the smaller diameter “end-cap” foam to dangle freely within the space. For example, in FIG. 9D the proximal portion has separated from the first large diameter plug and undesirably moved into the true lumen thereby obstructing blood flow. To prevent such a scenario, the smaller end-cap may be fixedly attached to the first large diameter plug or to a portion of a backbone.

FIGS. 10A-10B show an embodiment with even greater flexibility with respect to the embodiments of FIGS. 7A-9B due to its inclusion of voids or gaps where no foam is present between foams. FIG. 10A shows the implant before crimping and marker band (i.e., end cap) addition. In this embodiment, one or more of the SMP foams 1002, 1003, 1004, 1005, 1006 is slidably coupled to the backbone 1001 (e.g., platinum alloy wire for visualization). In an embodiment, some foams may be slidably coupled to the backbone (e.g., the middle three foams) while others are affixed to the backbone (e.g., the outer two foams). In other embodiments, all of the foams are slidably coupled to the backbone. In other embodiments, none of the foams are slidably coupled to the backbone (and instead are fixedly adhered or coupled to the backbone). The larger expanded diameter foams 1003, 1004, 1005 may be crimped into higher density foams located between the less densely crimped foams 1002, 1006. Gaps (e.g., gaps 1009, 1010) located between the foams help promote flexibility of the device during implantation and post implantation. Marker bands 1007, 1009 may be used to further facility visualization of the beginning and end of the foam series.

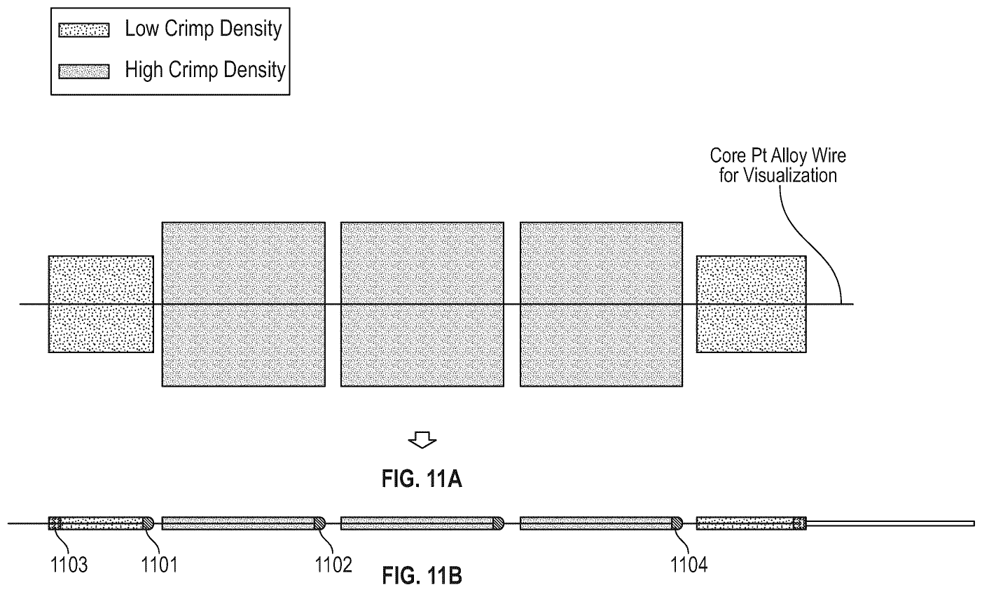

FIGS. 11A-11B are similar to FIGS. 10A-10B but include the use of element such as elements 1102, 1103. These elements may be adhesive and/or marker bands (e.g., 1102, 1103) to fix one end of one or more foams to the backbone, while leaving the opposing ends of such foams free to slide along the backbone. These may be coupled with other elements such as radiopaque marker bands 1103, 1104. As a result, the foams may be less likely to bunch about one another (because one end of each foam is statically coupled to the backbone), where bunching may be undesirable in some applications. However, such bunching may be desirable in other applications. For example, bunching of foams about an entry to a void (e.g., aortic aneurysm) may be deemed beneficial by some clinicians. Adhesive may include UV adhesive.

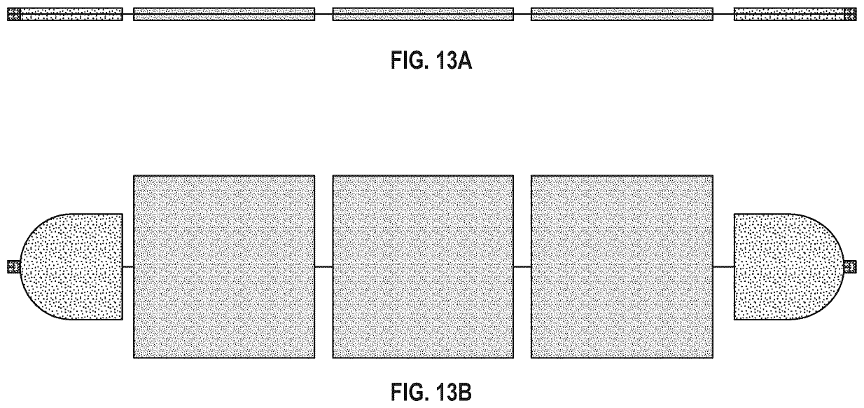

FIGS. 12B and 13B illustrate how adhesive and/or marker bands (e.g., 1202, 1203) cause the foams to narrow toward the adhesive and/or marker band. Adhesive and/or marker bands on both sides of a SMP foam may cause the foam to look ovular or somewhat spherical (see, e.g., left most foam in FIG. 12B). In the case of FIG. 12B, proximal fixation of each individual foam section enables more consistent device retrieval into the delivery catheter without shearing the foam relative to the core wire. In FIG. 12B, elements 1201, 1202, 1205 may be adhesive and/or marker bands to fix one end of one or more foams to the backbone, while leaving the opposing ends of such foams free to slide along the backbone.

These may be coupled with other elements such as radiopaque marker bands 1203, 1204. Similar to other embodiments addressed above, the embodiments of FIGS. 12B and 13B may have more densely crimped foams 1207, 1208, 1209 in combination with less densely crimped foams 1206, 1210.

In an embodiment, marker bands can be crimped, swaged, bonded or otherwise affixed directly to the core wire. This allows for full foam expansion (maximum expanded foam volume). In such an embodiment, one or more foams may be slidably coupled between such marker bands or fixedly attached between such marker bands. Placement of marker bands with or without UV adhesive (e.g., see FIG. 11B) may define/fix the axial position of the implants. In contrast, other embodiments addressed herein have foams allowed to freely float on the core wire which may allow the plugs to bunch and agglomerate to one side (which may be a disadvantage or advantage depending on clinical factors).

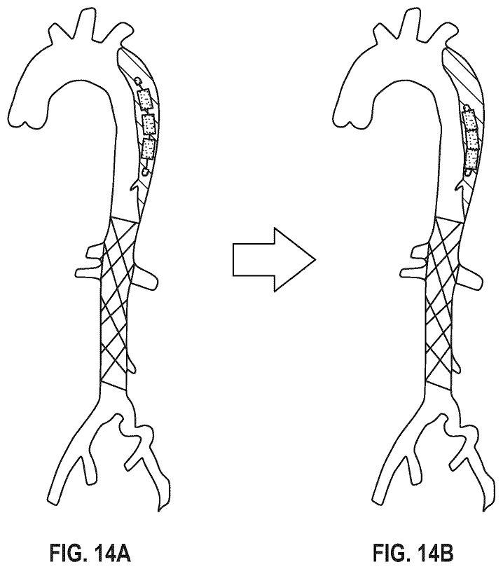

FIGS. 14A-14B address a system and method of use thereof. In the embodiment the advantages of flexibility during implantation (due to spacing between foams) are combined with stability after implantation (due to lack of spacing between foams). The core wire may be shortened in various ways. For example, the backbone may be formed of a polyurethane shape memory polymer foam that contracts axially and expands radially upon activation. Other ways to shorten the backbone include the following. First, the backbone is a shape memory polymer filament that axially contracts when exposed to heat, laser irradiation, or some other external stimulus. Second, the backbone is a filament (metal, polymer, or woven textile) that is pulled through a proximal cinching mechanism (e.g., incorporated into the marker band, or as a stand-alone component). The cinching component allows the backbone member to slide proximally, but restricts the filament from moving distally. At least some of the foam segments along the backbone are slidably coupled to the backbone and axially compress together when the backbone filament is shortened. Third, the backbone is a filament (metal or polymer) that is pulled through a proximal aperture member on the implant (either incorporated into the marker band, or as a stand-alone component). The filament can freely slide proximally and distally within this aperture member, until the aperture is activated (e.g., a mechanical channel may be closed) to lock the filament in place. Once activated the filament can no longer move distally within the aperture member. In some embodiments, the aperture member still allows the filament to move proximal for further axial shortening of the implant after the aperture member has been activated. Fourth, in some embodiments, the length of backbone filament that has been pulled proximally through the cinching and/or aperture member can be trimmed from the final implant. The filament can be trimmed by the detachment mechanism that deploys the implant from the proximal delivery pushwire (e.g., a heating element that melts the backbone filament, or an electrode that electrolytically degrades a stainless-steel core filament). Alternatively, the external core filament-trimming energy can be delivered by the delivery catheter (e.g., heating, electrolytic, radio frequency, laser, or other forms of energy). Fifth, in other embodiments, the filament wire that is pulled proximally through the cinching or proximal aperture member remains attached to the implant and flows freely in the implanted vessel anatomy while attached to the implant. Sixth, in other embodiments, the filament wire that is pulled proximally through the cinching or proximal aperture member remains attached to the implant and has a pre-shaped geometry that mitigates the amount of free space occupied by the filament backbone. In an example, the free section of proximally pulled filament that is not contained within a shape memory polymer foam coils to form a high surface area to volume ratio within the target vessel anatomy.

Thus, in FIGS. 14A-14B following initial deployment and foam expansion, the flexible core wire may be shortened to reduce or eliminate gaps between individual implant segments. This feature may be utilized to facilitate improved stability of the implant assembly within the aneurysm/luminal space.

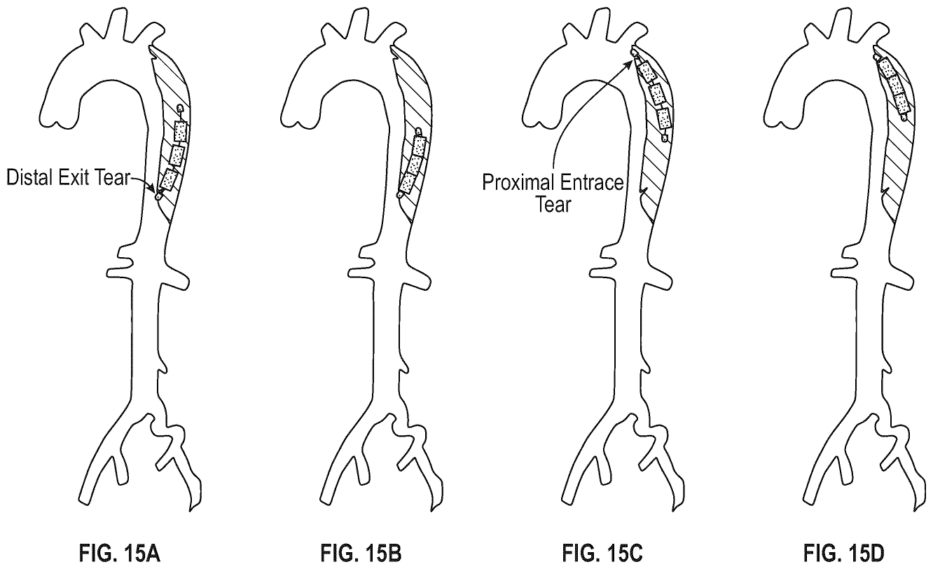

FIGS. 15A-15D address a method whereby the axial shortening system may be deployed alone or, for example, after other implants have already been implanted into the anatomical void (e.g., false lumen). The shortening embodiment may be used to better seal the void entrance. Thus, the axial shortening feature of the device may be employed to seal a tear that serves as a flow entrance to the dissection (proximal tear). Axial cinching/shortening may also be employed for distal tear sealing. Tear sealing may be a feature of the false luminal embolization implant, or be a separate adjunct to the luminal embolization device.

FIGS. 16A-16B address how the torquing feature of, for example, FIG. 5 may be utilized to facilitate embolic device (e.g., shape memory polymer foam, hydrogel) entanglement. Following foam expansion, the individual implants disposed along the flexible core wire may become entangled, yielding a structure which is more stable within the aneurysm space (e.g., less likely to migrate). The entanglement may be facilitated through torquing the assembly during delivery (as previously disclosed). Entanglement may also occur in a random manner as the implant assembly is advanced or packed into the aneurismal space. Entanglement may also be facilitated by imposing pre-determined curves or other shapes in the core wire. If the core wire is constructed of a shape memory material such as Nitinol, the implant assembly may be delivered to the target location via catheter in a nominally straight configuration, but then assume a secondary configuration upon deployment with the blood vessel or aneurysm. Secondary backbone shape may also be formed through elastic recovery of an annealed platinum alloy coil.

An embodiment may include a multi-splined device. For example, two backbones may each affix to the same single proximal endcap (such as a marker band) and the same single distal endcap (such as a marker band). The backbones may include memory, such as a shape memory polymer, Nitinol, and the like. After deployment from a delivery conduit, the backbones may retract axially and arc away from each other to form an umbrella of struts/backbones that better help rapidly deploy a large number embodiments elements (e.g., 3-5 elements per backbone) into a void.

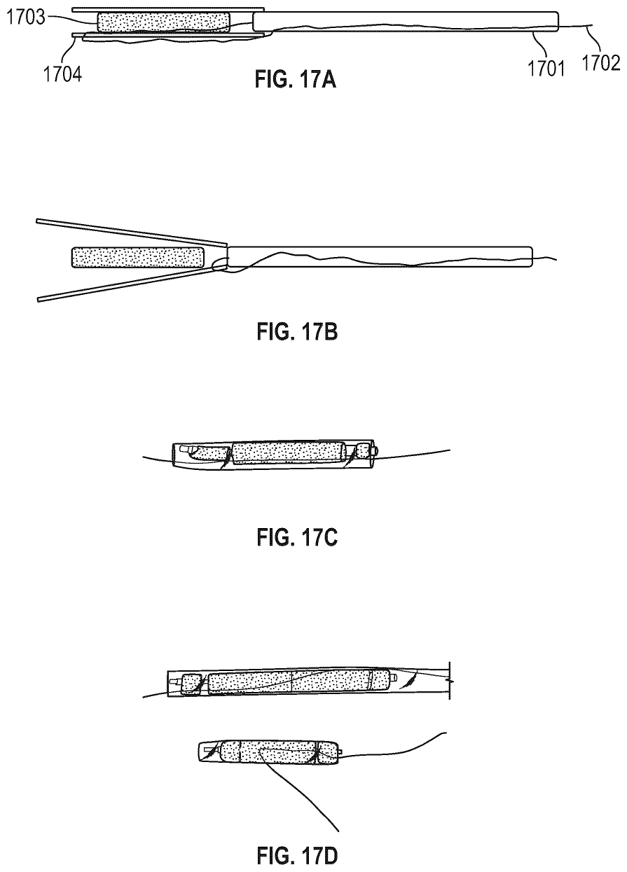

In FIGS. 17A-17D, embodiments may include a thin-walled polymer tubing 1704 placed over the crimped SMP foam implant 1703 to help retain the compressed outer diameter (OD) during packaging, shipping, and storage. The polymer tubing may include polyethylene terapthalate (PET), PEBAX, or another extrudable thermoplastic material. Following extrusion, the material may be radially blown with compressed gasses to expand the radial dimension and effect a thinner wall (e.g., 0.0005″ to 0.0015″). The extruded/blown material may have high radial strength to help maintain the diameter of the crimped SMP foams. Through any combination of (1) material selection, (2) appropriate material thickness, (3) extruding under axial strain to axially align polymer chains, (4) perforations along the finished tubing axis, and/or (5) other parameters in the extraction/blowing process, one can bias the material sleeve to tear longitudinally along the axis of the tubing to facilitate removal of the sleeve. A thin piece of thread or suture (or similar high tensile strength material) 1702 may be used to help propagate a longitudinal tear for removal of the sleeve (FIGS. 17A-17D). In one embodiment, the tear-away sleeve can be utilized for packaging/storage only and removed by the operator immediately prior to device introduction into the patient. In another embodiment, the tear-away sleeve could be configured to be part of the delivery and implant release system. In such an embodiment, the proximal end of the tear-away tubing is secured to a delivery wire or shaft (e.g., shaft 1701). During device manufacturing, the sleeve may be radially shrunk onto the foam plug, or the foam may be radially expanded into the sleeve. In either case, the foam becomes secured within the tear-away sleeve while also capturing a thread for tearing the tubing (FIGS. 17A, 17C). The foam plug (secured within the tear-away sleeve) is advanced to the desired anatomical location for release. The tear-away sleeve is removed by proximally retracting the captured suture in a way that cuts through the wall of the tear-away sleeve (FIGS. 17B, 17D). The tear away sleeve is still coupled to the delivery wire, and is removed from the patient with the delivery system, leaving only the embolic implant in place.

Embodiments may include implants with radiopaque markers for intra and post-procedure monitoring of device placement via x-ray imaging. Other embodiments include implants that have no radiopaque features to reduce imaging artifacts with later diagnostic imaging systems such as CT scans. These device embodiments can be visualized intra and post procedure with ultrasound or optical coherence tomography (OCT) imaging modalities. Multi-plug devices can be linked with polymeric (biodurable or biodegradable) carrier members. These embodiments may provide 100% degradation after implantation.

In an embodiment of a method, non-radiopaque device embodiments may be delivered into the false lumen of the AD while a second intravenous ultrasound (IVUS) catheter is placed in the true lumen of the aorta. This method allows one to visualize the false lumen anatomy, monitor device positioning, and monitor the SMP foam expansion independent of angiography (resulting in less radiation and a lower volume of injected contrast agent). The ultrasound imaging can be used independent of fluoroscopy, or in conjunction with x-ray imaging modalities.

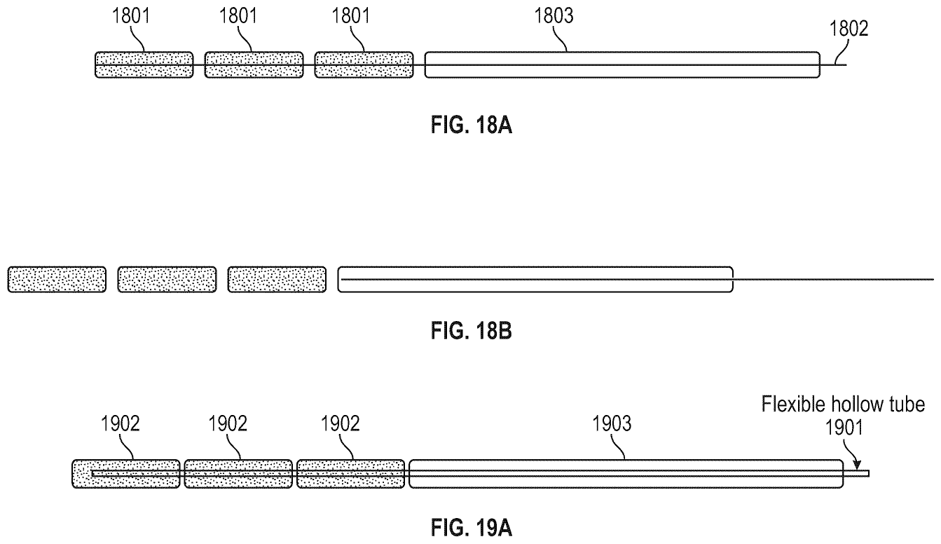

In another embodiment, the delivery system includes a radiopaque element 1802 such as a platinum-iridium or platinum-tungsten alloy wire that is threaded through the full length of the embolic implant (single foam plug or multiple foam plugs 1801) as seen in FIG. 18A. If multiple SMP foams are used, they be coupled to one another via a thread or other backbone. However, in other embodiments the foams are free from one another once member 1802 is removed. During implant delivery, the wire will be visible under fluoroscopy and will help define the location of the foam. This radiopaque element can be withdrawn from the implant as part of the implant release mechanism, or independently from device detachment from the delivery system (FIG. 18B, including delivery shaft 1803). After retraction, the implant can be entirely radiolucent to minimize subsequent x-ray or CT imaging artifacts, or the implant can include additional radiopaque elements for device identification in x-ray imaging.

Thus, for FIGS. 18A-18B the delivery system includes a radiopaque element such as a Pt/Ir wire, which can be threaded through the length of the implant. During implant delivery, the wire will be visible under fluoroscopy and will help to define location of the foam. Following release of the foam implant from the delivery catheter, the radiopaque wire is withdrawn, leaving only foam behind. This embodiment may be combined with the tear-away sleeve delivery/implant release concept of FIGS. 17A-17D.

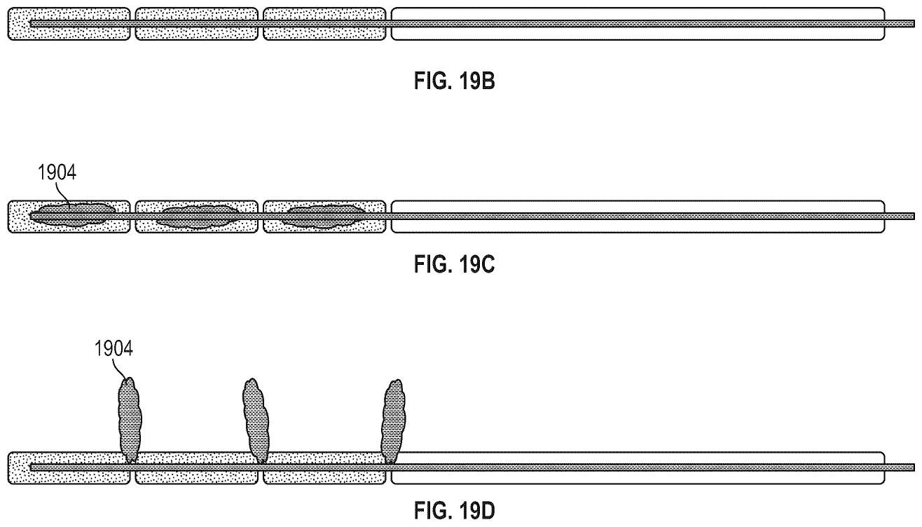

In another embodiment, a flexible tube 1901 is threaded through one or more of the crimped expansile plugs (e.g., SMP foam, hydrogel) 1902 during delivery using shaft 1903, as seen in FIGS. 19A-19D. The tubing 1901 may be stainless steel, nitinol, a single or multilumen polymer extrusion, or a braid or coil reinforced polymer tube such as polyimide. Contrast 1904 may be delivered through the tubing for visualization. For example, in FIG. 19B tube 1901 is filled with contrast to enable visualization of the system. Contrast injected through the tube will render the tubing radiopaque providing information on the device location and plug orientation. Fenestrations in the tubing (e.g., laser machined or loose sections of coil/braiding) can facilitate delivery of liquid contrast agent directly into the foam (while crimped and/or after SMP foam expansion) for improved visualization of the plugs (FIG. 19C). Further, the tubing fenestrations may be located between the foam plugs to enable contrast injection into the false lumen anatomy (FIG. 19D) (while crimped and/or after SMP foam expansion). Prior to device delivery into the patient, the tubing may be flushed with sterile saline to remove all air. Following release of the embolic implant, the tube may be withdrawn, leaving only foam behind.

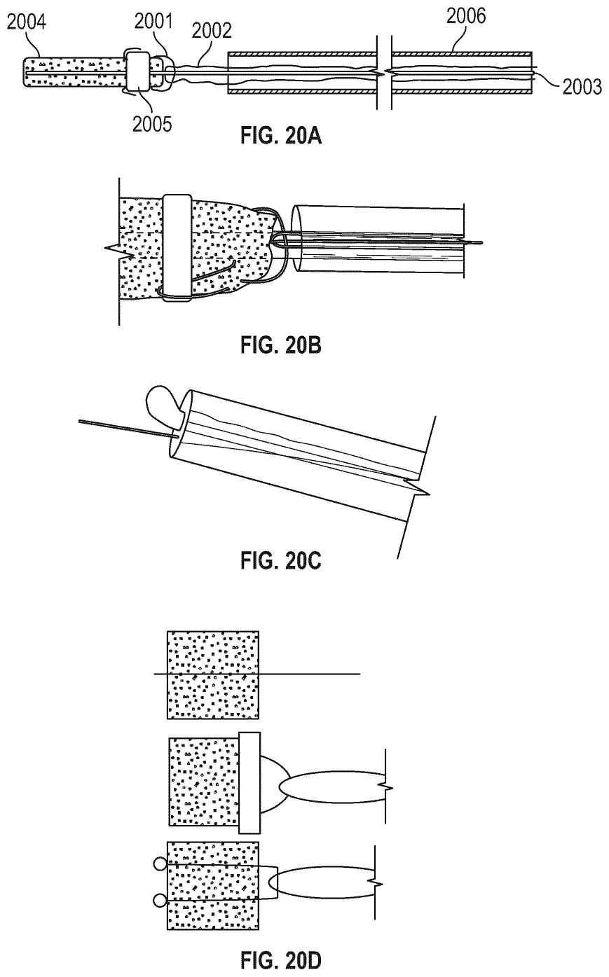

Embolic implant detachment from the delivery system may be achieved with, for example, a pin and loop release mechanism outlined in FIGS. 20A-20D. This mechanism includes, for example, (a) an implant-side (e.g., SMP foam 2004) retaining loop 2001 that is ultimately implanted in the patient, (b) a retaining loop 2002 on the delivery system side (which may include delivery catheter shaft 2006) that is ultimately removed from the patient, and (c) a retractable core wire 2003 that is removed from the patient. The closed end of the implant and delivery system retaining loops are positioned to overlap and the distal portion of the retractable core wire is threaded between the two closed loops and advanced into the implant, effectively capturing and holding the implant. To release the implant, the core wire is retracted so the distal tip is proximal to the implant retaining loop. Marker band 2005 may aid visualization.

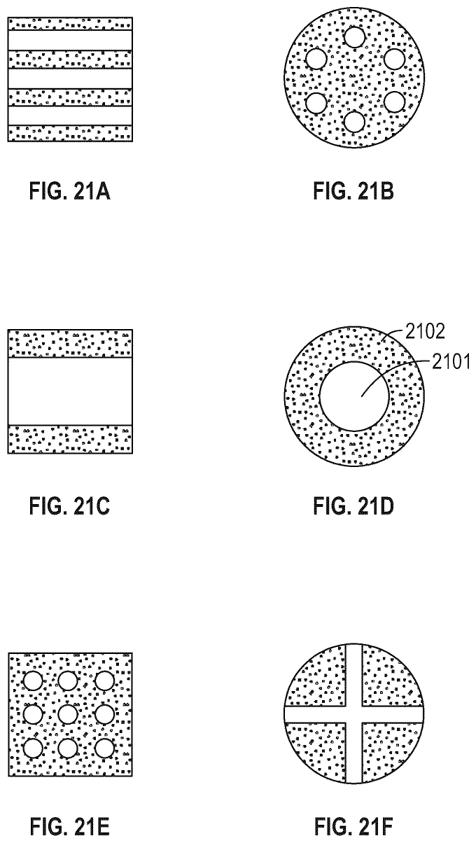

Embodiments may include large diameter embolic plugs (20 mm and greater) with a uniform foam density and volume, as in a solid cylinder of uniform foam. Other embodiments may have a gradient in foam density as a result of the foam synthesis parameters. Other embodiments may have variable foam density due to foam volumes removed from the bulk plug during manufacturing (see FIGS. 21A-21F). Material can be removed as cores along the axis of the foam cylinder, spokes removed orthogonal to the cylinder axis, cores directly removed from the central foam axis, or combinations thereof. Selective removal of material from the foam volume enables a higher crimp ratio (i.e., smaller crimped diameter for equivalent expanded diameter). A higher crimp ratio maximizes the volumetric expansion a device can achieve after being delivered through a small cross section delivery catheter for minimally invasive device delivery. Embodiments of foam plugs can still fully expand with selective removal of material, and other embodiments with significant foam volume removal can incorporate other features (e.g., shape memory alloy splines) that supplement foam expansion once the foam goes through the glass transition temperature and becomes rubbery. Below the glass transition temperature, the compressed foam material is stiff enough to constrain the shape memory alloy splines and maintain a crimped, smaller diameter cross section. For example, SMP foam or Nitinol wires may be included within a void (e.g., void 2101) to help expand the outer foam 2102.

Device features including plug entanglement, hooks that integrate with the vessel wall, and/or tissue adhesives coating the outer diameter of the crimped plug that adhere the device to the vessel wall are intended to keep the implant device within a target localized volume of the false lumen. Control over the anatomical positioning of the embolic implant within the false lumen space allows for selective embolization of the AD while avoiding collateral vessels that are connected to the AD anatomy that should also remain patent. Alternative embodiments use bulk foam plugs that are locally positioned with the delivery catheter and held in place until full expansion and the thrombus integrated with the expanded foam provides adhesion to local tissues. Once the expanded device is confidently held in place, the device is detached and the delivery system is removed from the patient.

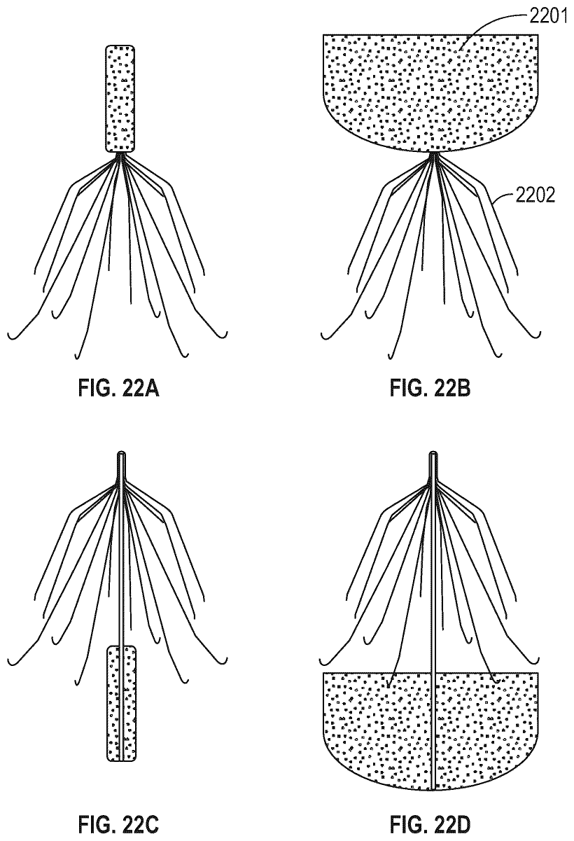

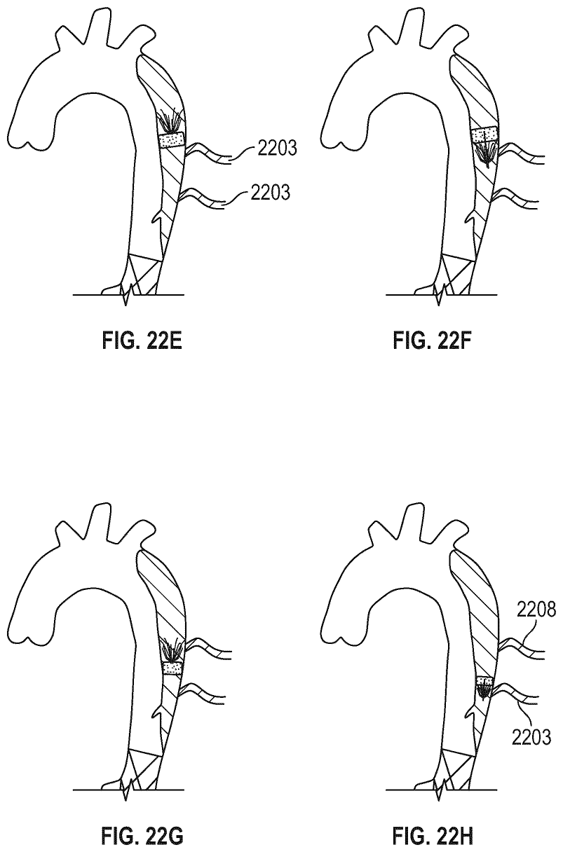

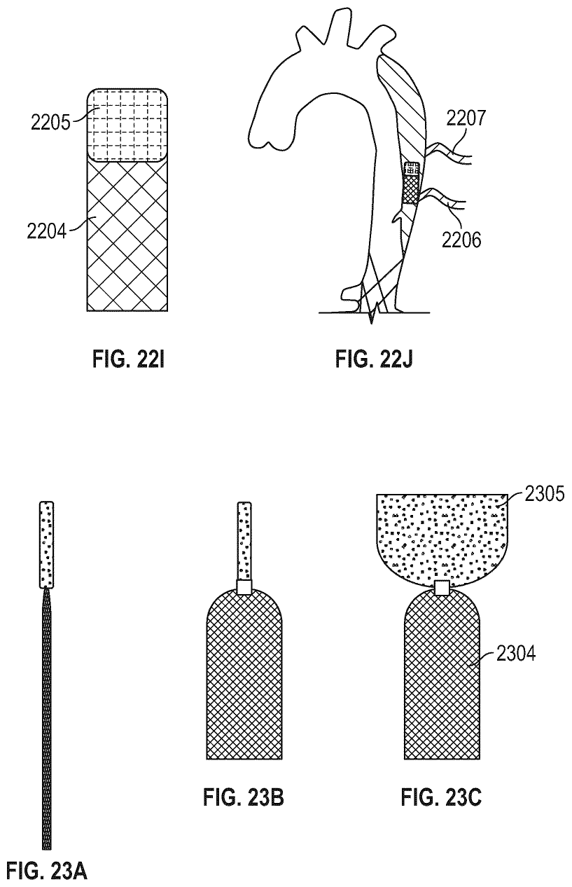

For example, in FIGS. 22A-22D depict a SMP foam 2201 coupled to 2202 inferior vena cava (IVC) filter. The IVC filter (or other anchor member) includes radially expanding barbs on the distal-facing portion of implant. The proximal-facing portion of the implant comprised of the SMP foam. The SMP foam plug will block/inhibit blood flow and the barbs help to stabilize and center the foam within the false lumen. This may reduce the volume of foam required to block or obstruct blood flow.

FIGS. 22E-22H shows a method of implanting the system of FIGS. 22A-22D. Under fluoroscopy, a user may position the implant such that the expanded foam will not block orifices of communicating vessels. Vessels 2203 located distal to (below) the plug will receive blood feeding up from the re-entry tear. Blood flow to vessels 2208 which originate above the plug may still receive limited flow from blood that flows past the plug. Over time, thrombus and tissue ingrowth will further limit (and potentially eliminate flow past the plug). During this time, the body would have time to accommodate and potentially establish flow via collateralization.

FIGS. 22I-22J shows a system similar to that of FIG. 22A but use a stent or skeletal system 2204 coupled to a SMP foam 2205. The stent may be nominally cylindrical in shape but able to conform to the shape of the false lumen. The foam element is coupled to the stent. The foam may be positioned fully within the stent, partially within the stent, or fully outside of the stent (but attached to the stent, for example via a tether wire or swaged marker band). The stent helps to stabilize and center the foam within the false lumen and may reduce the volume of foam required to block or obstruct blood flow. Under fluoroscopy, user positions the implant such that expanded foam will not block orifices of communicating vessels. Vessels 2203 located distal to (below) plug will receive blood feeding up from re-entry tear. Blood flow to vessels 2208 which originate above the plug may still receive limited flow from blood that flows past the plug. Over time, thrombus and tissue ingrowth will further limit (and potentially eliminate flow past the plug). During this time, the body would have time to accommodate and potentially establish flow via collateralization. The stent mesh size can be tuned to help occlude collateral vessels (fine mesh) or help them remain patent, even when the stent crosses the vessel branch point (loose mesh).

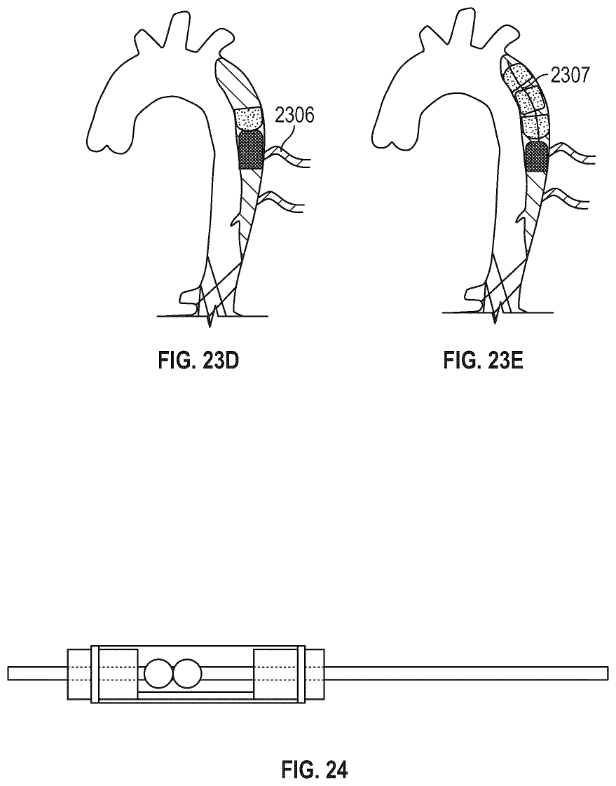

FIGS. 23A-23C show a SMP foam 2305 coupled to a distal end of a skeletal support system (e.g., stent 2304). In FIG. 23D the stent is placed over vessels 2306. FIG. 23E discloses a variation of the embodiment FIG. 23A, albeit with several embolic devices distally coupled to the stent via a tether 2307. Tether 2307 may traverse through the middle of the three foams (note: FIG. 23E shows a cross-sectional view so the tether is visible even though it traverses the middle of the foams).

The following examples pertain to further embodiments.

### First Example Set

Example 1. A system comprising: a backbone; a first polyurethane shape memory polymer (SMP) foam on the backbone; a second polyurethane SMP foam on the backbone; a third polyurethane SMP foam on the backbone and between the first and second SMP foams.

The backbone may be stiff or flexible. The backbone may include a metal, polymer, suture, thread, woven textile, and the like.

Example 2. The system of example 1, comprising first and second radiopaque conduit portions on the backbone, wherein the first, second, and third SMP foams are between the first and second radiopaque conduit portions.

Example 3. The system according to any of examples 1-2, wherein at least one of the first, second, and third SMP foams is slidably coupled to the backbone.

For example, see the slidable “pearls on a string” of FIG. 6C.

Example 4. The system of example 3, wherein at least another one of the first, second, and third SMP foams is statically and non-slidably coupled to the backbone.

Example 5. The system according to any of examples 1-2, wherein the at least one of the first, second, and third SMP foams includes a first portion that is slidably coupled to the backbone and a second portion that is statically coupled to the backbone.

Example 6. The system according to any of examples 1-5, wherein the third SMP foam includes a counter-bore portion to enclose at least a portion of the first SMP foam.

Example 7. The system of example 6, wherein: the backbone is oriented along a long axis that traverses the first, second, and third SMP foams; first and second planes are orthogonal to the long axis; the first plane intersects the first and third SMP foams; the second plane intersects the third SMP foam but not the first SMP foam.

Example 8. The system of example 2, wherein: the backbone is oriented along a long axis that traverses the first, second, and third SMP foams; a plane is orthogonal to the long axis; the plane intersects the first SMP foam and the first radiopaque conduit portion.

For example, see FIG. 12B.

Example 9. The system of example 8, wherein the first SMP foam includes an outer diameter that narrows as it approaches the first radiopaque conduit portion and when the first SMP foam is expanded.

Example 10. The system according to any of examples 1-9, wherein: each of the first, second, and third SMP foams includes an expanded primary shape and a compressed secondary shape; the first SMP foam includes a first maximum outer diameter in its primary shape; the second SMP foam includes a second maximum outer diameter in its primary shape; the third SMP foam includes a third maximum outer diameter in its primary shape; the third maximum outer diameter is greater than at least one of the first or second maximum outer diameters in their primary shapes.

For example, see FIGS. 6A, 7B, or 12B.

Example 11. The system of example 10, wherein: the first SMP foam includes a first maximum outer diameter in its secondary shape; the second SMP foam includes a second maximum outer diameter in its secondary shape; the third SMP foam includes a third maximum outer diameter in its secondary shape; the third maximum outer diameter is equal to each of the first and second maximum outer diameters in their secondary shapes.

See, for example, FIG. 7C or 12A.

Example 12. The system of example 11, wherein: the first SMP foam includes a first crimp density in its secondary shape; the second SMP foam includes a second crimp density in its secondary shape; the third SMP foam includes a third crimp density in its secondary shape; the third crimp density is greater than the first crimp density.

Example 13. The system according to any of examples 1-12, wherein the backbone is configured to shrink axially in response to stimulus, the stimulus including at least one of heat, light, moisture, magnetic energy, or combinations thereof.

In an embodiment, the backbone may have first and second configurations. For example, the backbone may include Nitinol and transform from a first configuration (that is generally linear to fit in a delivery conduit and be passed through vasculature) to a second configuration (that is coiled into a helical shape, conical shape and the like). During the transition between the first and second configurations, the backbone may decrease in length from its proximal most tip to its distal most tip.

Example 14. The system according to any of examples 1-13 comprising a mechanical lock coupled to the backbone, wherein the mechanical lock is configured to resist distal movement of the backbone.

Example 15. The system of example 14, wherein the mechanical lock is configured to allow proximal movement of the backbone.

Example 16. The system according to any of examples 14-15, wherein the mechanical lock includes a cinch.

Example 17. The system according to any of examples 1-16 comprising an additional backbone that traverses the first, second, and third SMP foams.

For example, see FIG. 5A.

Example 18. The system of example 17, wherein the additional backbone is primarily parallel to the backbone.

Example 19. The system according to any of examples 17-18, wherein: the third SMP foam includes a first portion and a second portion; the first portion of the third SMP foam is stiffer than the second portion of the third SMP foam; the additional backbone is slidingly coupled to the first portion of the third SMP foam.

For example, for the stiffer first portion of the third SMP foam see the “internal torque member” of FIGS. 5A-5D.

Example 20. The system of example 19 comprising a fourth SMP foam between the second and third SMP foams, wherein: the fourth SMP foam includes a first portion and a second portion; the first portion of the fourth SMP foam is stiffer than the second portion of the fourth SMP foam; the additional backbone is slidingly coupled to the first portion of the fourth SMP foam.

Example 21. The system of example 20, wherein: in a first orientation the additional backbone is slidingly coupled to the first portion of each of the third and fourth SMP foams; in a second orientation the additional backbone is retracted and slidingly coupled to the first portion of the third SMP foam but is no longer slidingly coupled to the fourth SMP foam.

For example, see FIGS. 5B-5C.

Example 22. The system according to any of examples 17-21, wherein the first portion of the third SMP foam includes a SMP foam.

Example 23. The system according to any of examples 17-22, wherein: the additional backbone couples to the first portion of the third SMP foam at a first location; the additional backbone at the first location has a non-circular cross-section; the non-circular cross-section is oriented orthogonal to a long axis of the additional backbone.

Example 24. The system according to any of examples 19-23, wherein the second portion of the third SMP foam surrounds the first portion of the third SMP foam in a plane that is orthogonal to a long axis of the additional backbone.

Example 25. The system according to any of examples 1-24, wherein: the backbone is oriented along a long axis that traverses the first, second, and third SMP foams; a short axis is orthogonal to the long axis; the third SMP foam includes a contiguous outer surface that surrounds the third SMP foam in a plane that is orthogonal to the long axis; the third SMP foam includes a first void that includes no foam cells; the short axis intersects the first void; and the short axis intersects the contiguous outer surface of the third SMP foam at least twice.

For example, see void 2101 of foam 2102 in FIG. 21D.

Example 26. The system according to any of examples 25, wherein: an additional short axis is orthogonal to the long axis; the first SMP foam includes a second void that includes no foam cells; the additional short axis intersects the second void; and the additional short axis intersects the contiguous outer surface of the first SMP foam at least twice.

Example 27. The system according to any of examples 1-24, wherein: the backbone is oriented along a long axis that traverses the first, second, and third SMP foams; a short axis is orthogonal to the long axis; the first SMP foam includes a contiguous outer surface that surrounds the first SMP foam in a plane that is orthogonal to the long axis; the first SMP foam includes a first void that includes no foam cells; the short axis intersects the first void; and the short axis intersects the contiguous outer surface of the first SMP foam at least twice.

Example 28. The system according to any of examples 27, wherein: an additional short axis is orthogonal to the long axis; the first SMP foam includes a second void that includes no foam cells; the additional short axis intersects the second void; and the additional short axis intersects the contiguous outer surface of the first SMP foam at least twice.

Example 29. The system according to any of examples 25-28, wherein: the system includes a resilient member; the short axis intersects the resilient member.

For example, the system may include a Nitinol arm that is included in the void and helps expand the foam.

Example 30. The system according to any of examples 1-29 comprising a structural support skeleton coupled to the second SMP foam, wherein: the second SMP foam is between the third SMP foam and the structural support skeleton.

For example, see FIG. 23E. The skeleton may include an IVC filter and/or a stent.

In an embodiment the backbone includes tether 2307 of FIG. 23E.

An embodiment may include any of the above examples albeit modified to include single SMP foam coupled to the skeleton. For example, see FIG. 23D.

An embodiment may include any of the above examples albeit modified to omit the backbone. For example, see FIG. 23C.

Example 31. A system comprising: a first polyurethane shape memory polymer (SMP) foam; a structural support skeleton coupled to the first SMP foam, the structural support skeleton including first and second opposing ends; wherein the first SMP foam is coupled to one of the first or second opposing ends.

For example, see FIG. 22A or 23C.

Example 32. A method comprising implanting at least a portion of the system according to any of examples 1-31 in a false lumen of an aortic dissection.

### Second Example Set

Example 1. A system comprising: a backbone; a first polyurethane shape memory polymer (SMP) foam on the backbone; a second polyurethane SMP foam on the backbone; a third polyurethane SMP foam on the backbone and between the first and second SMP foams.

See, for example, first SMP foam 1210, second SMP foam 1206, and third SMP foam 1209. Foam 1206 may be most distal and configured to emerge from a deliver conduit before either of foams 1209, 1210.

Example 2. The system of Example 1, comprising first and second radiopaque conduit portions on the backbone, wherein the first, second, and third SMP foams are between the first and second radiopaque conduit portions.

As a result, a user may be able to visualize the beginning and end of the system.

Another version of Example 2. The system of Example 1, comprising first and second radiopaque portions on the backbone, wherein the first, second, and third SMP foams are between the first and second radiopaque portions.

Thus, in some embodiments the radiopaque portion is not necessarily a conduit but instead may be, for example, a bead fixedly coupled (e.g., via weld or adhesive) to the backbone.

Another version of Example 2. The system of Example 1, comprising first and second radiopaque conduit portions on the backbone, wherein the third SMP foam is between the first and second radiopaque conduit portions.

For example, the first and second radiopaque conduit portions on the backbone may be affixed to proximal portions of the first and second SMP foams so that not all of the, for example, second foam is between the first and second radiopaque conduit portions.

Example 3. The system of Example 2, wherein at least one of the first, second, and third SMP foams is slidably coupled to the backbone.

Example 4. The system of Example 3, wherein at least another one of the first, second, and third SMP foams is statically and non-slidably coupled to the backbone.

For example, in an embodiment the end foams may be fixedly coupled to the backbone while the middle foam (or middle foams in an embodiment with two or more foams between the two outer foams) will be able to slide along the length of the backbone.

Example 5. The system of Example 3, wherein the at least one of the first, second, and third SMP foams includes a first portion that is slidably coupled to the backbone and a second portion that is statically coupled to the backbone.

For example, the most proximal foam may have its proximal end fixed to the backbone. For instance, a radiopaque marker may crimp over the proximal portion of the foam and hold the proximal portion of the foam statically against the backbone. Adhesive may or may not be used to hold the proximal portion of the foam against the backbone. By locating a portion of the foam in a radiopaque conduit/marker or coupled to the conduit/marker may cause the proximal portion of the foam to taper and facilitate withdrawal of the foam into a conduit (e.g., retrieving a system back into the deliver conduit to reposition the system). The distal portion of the proximal foam may be free to slide along the backbone. Therefore, the foam may expand radially and contract axially as transitions from its smaller diameter secondary form to its larger diameter expanded primary form.

Example 6. The system of Example 3, wherein: the first SMP foam includes a first portion that is slidably coupled to the backbone and a second portion that is statically coupled to the backbone; the second SMP foam includes a first portion that is slidably coupled to the backbone and a second portion that is statically coupled to the backbone.

Example 7. The system of Example 5, wherein the third SMP foam includes a first portion that is slidably coupled to the backbone and a second portion that is statically coupled to the backbone.

Example 8. The system of Example 5, wherein the third SMP foam is slidably coupled to the backbone.

Example 9. The system according to any of Examples 2-8, wherein: the backbone is oriented along a long axis that traverses the first, second, and third SMP foams; a plane is orthogonal to the long axis; the plane intersects the first SMP foam and the first radiopaque conduit portion.

Example 10. The system of Example 9, wherein the first SMP foam includes an outer diameter that narrows as it approaches the first radiopaque conduit portion and when the first SMP foam is expanded.

Example 11. The system of Example 10, wherein: an additional plane is orthogonal to the long axis; the additional plane intersects the second SMP foam and the second radiopaque conduit portion.

Example 12. The system of Example 11 comprising a third radiopaque conduits, wherein: another plane is orthogonal to the long axis; the another plane intersects the third SMP foam and the third radiopaque conduit portion.

For example, see FIG. 12B.

Example 13. The system of Example 9, wherein: the third SMP foam does not directly contact the first SMP foam; the third SMP foam does not directly contact the second SMP foam.

For example, spaces may exist between the foams to facilitate flexibility of the system and thereby facility navigation through vasculature. See, for example, FIG. 12B.

Example 14. The system of Example 9, wherein: each of the first, second, and third SMP foams includes an expanded primary shape and a compressed secondary shape; the first SMP foam includes a first maximum outer diameter in its primary shape; the second SMP foam includes a second maximum outer diameter in its primary shape; the third SMP foam includes a third maximum outer diameter in its primary shape; the third maximum outer diameter is greater than at least one of the first or second maximum outer diameters in their primary shapes.

Example 15. The system of Example 9, wherein: the first SMP foam includes a first maximum outer diameter in its secondary shape; the second SMP foam includes a second maximum outer diameter in its secondary shape; the third SMP foam includes a third maximum outer diameter in its secondary shape; the third maximum outer diameter is equal to each of the first and second maximum outer diameters in their secondary shapes.

Example 16. The system of Example 9, wherein the first SMP foam includes a polymer that comprises polymerized monomers, the monomers including hydroxypropyl ethylenediamine (HPED), triethanolamine (TEA), and hexamethylene diisocyanate (HDI).

Alternative version of Example 16. The system according to any of Examples 1-8, wherein the first SMP foam includes a polymer that comprises polymerized monomers, the monomers including: at least one of hydroxypropyl ethylenediamine (HPED), triethanolamine (TEA), or combinations thereof; at least one of hexamethylene diisocyanate (HDI), trimethyl hexamethylene diisocyanate, or combinations thereof.

Example 17. The system of Example 9 comprising an additional backbone that traverses the first, second, and third SMP foams.

Example 18. The system of Example 17, wherein at least one of the first, second, or third SMP foams includes an asymmetric cross-section taken parallel to the plane.

For example, see FIG. 3C. The asymmetric cross-section may help a user manage to occlude the false lumen while maintaining as wide as possible a passage for the true lumen of an AD.

Example 19. The system of Example 9, wherein: a short axis is orthogonal to the long axis; the third SMP foam includes a contiguous outer surface that surrounds the third SMP foam in an additional plane that is orthogonal to the long axis; the third SMP foam includes a first void that includes no foam cells; the short axis intersects the first void; and the short axis intersects the contiguous outer surface of the third SMP foam at least twice.

See, for example, FIGS. 21A, 21C, 21E.

Example 20. The system of Example 19, wherein the contiguous outer surface of the third SMP foam narrows as it slopes proximally.

Thus, the third SMP foam may have a “bullet” shape such as foam 1209 in FIG. 12E.

Example 21. The system of Example 19, wherein: an additional short axis is orthogonal to the long axis; the first SMP foam includes a second void that includes no foam cells; the additional short axis intersects the second void; and the additional short axis intersects the contiguous outer surface of the first SMP foam at least twice.

Example 22. The system of Example 9, wherein: a short axis is orthogonal to the long axis; the first SMP foam includes a contiguous outer surface that surrounds the first SMP foam in a plane that is orthogonal to the long axis; the first SMP foam includes a first void that includes no foam cells; the short axis intersects the first void; and the short axis intersects the contiguous outer surface of the first SMP foam at least twice.

Example 23. The system of Example 22, wherein the contiguous outer surface of the first SMP foam narrows as it slopes proximally.

Example 24. The system of Example 22, wherein: an additional short axis is orthogonal to the long axis; the first SMP foam includes a second void that includes no foam cells; the additional short axis intersects the second void; and the additional short axis intersects the contiguous outer surface of the first SMP foam at least twice.

Example 25. The system of Example 22, wherein: the system includes a resilient member; the short axis intersects the resilient member.

For example, the resilient member may be located within void 2101. The resilient member may be an arm attached to the backbone at one end, such as one of the arms of IVC filter 2202. The resilient member may include a shape memory alloy spline.

Example 26. The system according to any of Examples 1-8 comprising a structural support skeleton coupled to the first SMP foam, wherein: the first SMP foam is between the third SMP foam and the structural support skeleton.

In an embodiment, the skeleton may include a stent, multiarmed unit such as an IVC filter, a coil having a helical or conical shape, and the like.

Example 27. The system of Example 26 comprising a tissue adhesive coupled to the structural support skeleton.

In an embodiment, adhesive is located directly on the foam, instead of having a mechanical support skeleton or in addition to the skeleton. For example, any or all of the foams of FIG. 12B may include a tissue adhesive applied to portions of the foams. For example, the adhesive may be placed on one side of a proximal most foam. That part of the foam may be loaded with radiopaque particles to indicate its location so the user knows to apply the foam where the adhesive is located to the aortic wall. Such an embodiment may be coupled with a torque inducing backbone (e.g., see FIG. 5C) to better control location of the adhesive with respect to the aortic wall that is largely opposite the false lumen.

Example 28. The system of Example 27, wherein: the structural support skeleton has first and second sidewalls that oppose one another; the tissue adhesive is on the first sidewall but not the second sidewall.

For example, the skeleton may include a stent whose struts on one sidewall are coated with a tissue adhesive while the struts on the opposing sidewall are not coated with the tissue adhesive. In such a case, the non-coated sidewall could interface the false lumen and the adhesive coated sidewall could contact the aortic wall.

In another embodiment, the tissue adhesive could be substituted with another anchor of some sort. For example, only the first sidewall of the stent may include barbs to burrow into the aortic wall.

Example 29. A method comprising implanting at least a portion of the system according to any of Examples 9 or 26 in a false lumen of an aortic dissection.

Example 30. A method comprising implanting at least a portion of the system according to any of Examples 9 or 26 in a left atrial appendage (LAA).

### Third Example Set

Example 1. A system comprising: a backbone; a first polyurethane shape memory polymer (SMP) foam on the backbone; a second polyurethane SMP foam on the backbone.

Example 2. The system of example 1, comprising first and second radiopaque conduit portions on the backbone, wherein the first and second SMP foams are between the first and second radiopaque conduit portions.

Example 3. The system of example 2, wherein at least one of the first or second SMP foams is slidably coupled to the backbone.

Example 4. The system of example 3, wherein at least another one of the first or second SMP foams is statically and non-slidably coupled to the backbone.

Example 5. The system of example 3, wherein the at least one of the first or second SMP foams includes a first portion that is slidably coupled to the backbone and a second portion that is statically coupled to the backbone.

Example 6. The system of example 3, wherein: the first SMP foam includes a first portion that is slidably coupled to the backbone and a second portion that is statically coupled to the backbone; the second SMP foam includes a first portion that is slidably coupled to the backbone and a second portion that is statically coupled to the backbone.

Example 7. The system according to any of examples 1-6, wherein: the backbone is oriented along a long axis that traverses the first and second SMP foams; a plane is orthogonal to the long axis; the plane intersects the first SMP foam and the first radiopaque conduit portion.

Example 8. The system of example 7, wherein the first SMP foam includes an outer diameter that narrows as it approaches the first radiopaque conduit portion and when the first SMP foam is expanded.

Example 9. The system of example 8, wherein: an additional plane is orthogonal to the long axis; the additional plane intersects the second SMP foam and the second radiopaque conduit portion.

Example 10. The system of example 9 comprising a third radiopaque conduit portion, wherein: another plane is orthogonal to the long axis; the another plane intersects one of the first or second SMP foams and the third radiopaque conduit portion.

Example 11. The system of example 10, wherein the first SMP foam does not directly contact the second SMP foam.

Example 12. The system of example 1, wherein: each of the first and second SMP foams includes an expanded primary shape and a compressed secondary shape; the first SMP foam includes a first maximum outer diameter in its primary shape; the second SMP foam includes a second maximum outer diameter in its primary shape; the first maximum outer diameter is greater than the second maximum outer diameter.

Example 13. The system of example 1, wherein: the first SMP foam includes a first maximum outer diameter in its secondary shape; the second SMP foam includes a second maximum outer diameter in its secondary shape; the first maximum outer diameter is equal to the second maximum outer diameter.

Example 14. The system according to any of examples 1-13, wherein the first SMP foam includes a polymer that comprises polymerized monomers, the monomers including hydroxypropyl ethylenediamine (HPED), triethanolamine (TEA), and hexamethylene diisocyanate (HDI).

Example 15. The system according to any of examples 1-14, wherein: the backbone is oriented along a long axis that traverses the first and second SMP foams; a short axis is orthogonal to the long axis; the first SMP foam includes a contiguous outer surface that surrounds the first SMP foam in an additional plane that is orthogonal to the long axis; the first SMP foam includes a first void that includes no foam cells; the short axis intersects the first void; and the short axis intersects the contiguous outer surface of the first SMP foam at least twice.

Example 16. The system of example 15, wherein the contiguous outer surface of the first SMP foam narrows as it slopes proximally.

Example 17. The system of example 15, wherein: an additional short axis is orthogonal to the long axis; the first SMP foam includes a second void that includes no foam cells; the additional short axis intersects the second void; and the additional short axis intersects the contiguous outer surface of the first SMP foam at least twice.

Example 18. The system of example 15, wherein: the system includes a resilient member; the short axis intersects the resilient member.

Example 19. The system according to any of examples 1-18 comprising a structural support skeleton coupled to the first SMP foam, wherein: the first SMP foam is between the second SMP foam and the structural support skeleton.

Example 20. The system of example 19 comprising a tissue adhesive coupled to the structural support skeleton.

Example 21. The system of example 20, wherein: the structural support skeleton has first and second sidewalls that oppose one another; the tissue adhesive is on the first sidewall but not the second sidewall.

Example 22. A method comprising implanting at least a portion of the system according to any of examples 1-21 in a false lumen of an aortic dissection.

Example 23. A method comprising implanting at least a portion of the system according to any of examples 1-21 in a left atrial appendage (LAA).

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. A system comprising:
a backbone;
a first polyurethane shape memory polymer (SMP) foam on the backbone;
a second polyurethane SMP foam on the backbone;
a third polyurethane SMP foam on the backbone and between the first and second SMP foams.

2. The system of claim 1, comprising first and second radiopaque conduit portions on the backbone, wherein the first, second, and third SMP foams are between the first and second radiopaque conduit portions.

3. The system of claim 2, wherein at least one of the first, second, or third SMP foams is slidably coupled to the backbone.

4. The system of claim 3, wherein at least another one of the first, second, or third SMP foams is statically and non-slidably coupled to the backbone.

5. The system of claim 3, wherein the at least one of the first, second, and third SMP foams includes a first portion that is slidably coupled to the backbone and a second portion that is statically coupled to the backbone.

6. The system of claim 3, wherein:
the first SMP foam includes a first portion that is slidably coupled to the backbone and a second portion that is statically coupled to the backbone;
the second SMP foam includes a first portion that is slidably coupled to the backbone and a second portion that is statically coupled to the backbone.

7. The system of claim 5, wherein the third SMP foam includes a first portion that is slidably coupled to the backbone and a second portion that is statically coupled to the backbone.

8. The system of claim 5, wherein the third SMP foam is slidably coupled to the backbone.

9. The system of claim 2, wherein:
the backbone is oriented along a long axis that traverses the first, second, and third SMP foams;
a plane is orthogonal to the long axis;
the plane intersects the first SMP foam and the first radiopaque conduit portion.

10. The system of claim 9, wherein the first SMP foam includes an outer diameter that narrows as it approaches the first radiopaque conduit portion and when the first SMP foam is expanded.

11. The system of claim 10, wherein:
an additional plane is orthogonal to the long axis;
the additional plane intersects the second SMP foam and the second radiopaque conduit portion.

12. The system of claim 11 comprising a third radiopaque conduit portion, wherein:
another plane is orthogonal to the long axis;
the another plane intersects the third SMP foam and the third radiopaque conduit portion.

13. The system of claim 9, wherein:
the third SMP foam does not directly contact the first SMP foam;
the third SMP foam does not directly contact the second SMP foam.

14. The system of claim 9, wherein:
each of the first, second, and third SMP foams includes an expanded primary shape and a compressed secondary shape;
the first SMP foam includes a first maximum outer diameter in its primary shape;
the second SMP foam includes a second maximum outer diameter in its primary shape;
the third SMP foam includes a third maximum outer diameter in its primary shape;
the third maximum outer diameter is greater than at least one of the first or second maximum outer diameters in their primary shapes.

15. The system of claim 9, wherein:
the first SMP foam includes a first maximum outer diameter in its secondary shape;
the second SMP foam includes a second maximum outer diameter in its secondary shape;
the third SMP foam includes a third maximum outer diameter in its secondary shape;
the third maximum outer diameter is equal to each of the first and second maximum outer diameters in their secondary shapes.

16. (canceled)

17. The system of claim 9 comprising an additional backbone that traverses the first, second, and third SMP foams.

18. The system of claim 17, wherein at least one of the first, second, or third SMP foams includes an asymmetric cross-section taken parallel to the plane.

19. The system of claim 9, wherein:
a short axis is orthogonal to the long axis;
the third SMP foam includes a contiguous outer surface that surrounds the third SMP foam in an additional plane that is orthogonal to the long axis;
the third SMP foam includes a first void that includes no foam cells;
the short axis intersects the first void; and
the short axis intersects the contiguous outer surface of the third SMP foam at least twice.

20. The system of claim 19, wherein the contiguous outer surface of the third SMP foam narrows as it slopes proximally.

21. The system of claim 19, wherein:
an additional short axis is orthogonal to the long axis;
the third SMP foam includes a second void that includes no foam cells;
the additional short axis intersects the second void; and
the additional short axis intersects the contiguous outer surface of the third SMP foam at least twice.

22. The system of claim 9, wherein:
a short axis is orthogonal to the long axis;
the first SMP foam includes a contiguous outer surface that surrounds the first SMP foam in a plane that is orthogonal to the long axis;
the first SMP foam includes a first void that includes no foam cells;
the short axis intersects the first void; and
the short axis intersects the contiguous outer surface of the first SMP foam at least twice.

23. The system of claim 22, wherein the contiguous outer surface of the first SMP foam narrows as it slopes proximally.

24. The system of claim 22, wherein:
an additional short axis is orthogonal to the long axis;
the first SMP foam includes a second void that includes no foam cells;
the additional short axis intersects the second void; and
the additional short axis intersects the contiguous outer surface of the first SMP foam at least twice.

25-30. (canceled)
